(19) United States
(12) Patent Application Publication
(10) Pub. No.: US 2018/0313858 A1
(43) Pub. Date: Nov. 1, 2018

(54) QUANTIFICATION OF DRUGS IN BIOLOGICAL SAMPLES

(71) Applicant: Indivior UK Limited, Berkshire (GB)

(72) Inventors: Yongzhen Liu, Richmond, VA (US); Allan Xu, North Wales, PA (US); Xiaohua Li, North Wales, PA (US)

(21) Appl. No.: 15/772,457

(22) PCT Filed: Nov. 2, 2016
(86) PCT No.: PCT/IB2016/001718

§ 371 Date: Apr. 30, 2018

Related U.S. Application Data
(60) Provisional application No. 62/250,930, filed on Nov. 4, 2015.

(51) Int. Cl. G01N 33/94

(52) U.S. Cl. CPC G01N 33/9486

(57) ABSTRACT

The disclosure provides methods for the detection and quantification of drugs and drug metabolites in biological samples, such as naloxone, buprenorphine, and norbuprenorphine.

### CROSS-REFERENCE TO RELATED APPLICATION

This application claims priority to U.S. Application No. 62/250,930 filed Nov. 4, 2015, the disclosure of which is incorporated by reference herein in its entirety.

### FIELD

The disclosure provides methods of identifying and quantifying drugs and drug metabolites in biological samples.

### BACKGROUND

Opioid dependence is a neurobehavioral syndrome characterized by the repeated, compulsive seeking and use of an opioid despite adverse social, psychological, and/or physical consequences. According to a National Survey on Drug Use and Health, approximately 2 million people over the age of 12 were opioid dependent by the end of 2013. As a result, society is facing serious consequences, such as high morbidity and mortality rates, increased health care costs, and crime.

SUBOXONE® (buprenorphine/naloxone, Indivior UK Limited) has been approved for the treatment of opioid dependence as a sublingual tablet or film formulation containing buprenorphine and naloxone at a fixed ratio of 4:1. Buprenorphine is a partial agonist at the mu-opioid receptor and possesses K-opioid receptor antagonist properties. The partial agonist activity of buprenorphine paired with its slow dissociation from the mu-opioid receptor has been associated with lower propensity for addiction in general signs and symptoms of physical dependence and a milder withdrawal syndrome as compared to full opioid agonists. Buprenorphine undergoes N-dealkylation to norbuprenorphine. Norbuprenorphine is a weak opioid receptor agonist. Naloxone is an antagonist at mu, kappa, and delta opioid receptors and can produce opioid withdrawal effects in individuals physically dependent on opioids. Naloxone was added to buprenorphine in pharmaceutical products to deter parenteral misuse and abuse of buprenorphine.

For the treatment of opioid dependence, sublingual buprenorphine is typically given as a single daily dose ranging from 4 to 24 mg/day, with the recommended target buprenorphine dose being 16 mg/day. The lowest currently approved dosage strength of SUBOXONEE® is buprenorphine/naloxone in an amount of 2 mg/0.5 mg, which is used as both the initial dose administered and in titration during the induction and medical tapering phases. In order to support the formulation development, a clinical phase I trial was conducted to investigate the exposure of buprenorphine, norbuprenorphine and naloxone in healthy volunteers following sublingual administration of buprenorphine/naloxone in amounts of 2 mg/0.5 mg.

Previously, the lower limitation of quantification for buprenorphine and norbuprenorphine was only as low as 0.05 ng/mL and 0.03 ng/mL, respectively, utilizing gas chromatography associated with electron capture detectors and a mass spectrometry detector. These techniques require a time-consuming derivatization procedure.

To date, liquid chromatography-electrospray-tandem mass spectrometry (LC-ESI-MS/MS) methods, with their high selectivity and sensitivity, are the primary technique for quantitation of buprenorphine and its metabolites in a wide variety of matrices, including plasma. In comparison to the previously described methods, LC-MS/MS methods provide rapid analytical run time, good separations within compounds, and improved sensitivity and selectivity without the need for derivatization.

Of the available LC-MS methods applied to plasma, the most sensitive assay has a lower limit of quantification at 0.001 ng/mL for buprenorphine and 0.01 ng/mL for norbuprenorphine, but naloxone is not analyzed. Naloxone is more hydrophilic than buprenorphine. Due to the low naloxone dose (i.e., 0.5 mg for sublingual buprenorphine/naloxone formulations) and poor bioavailability of naloxone, detecting all three analytes and achieving a low lower limit of quantification (LLOQ) for naloxone is very challenging.

In order to determine the full pharmacokinetic profiles by capturing the concentrations for all three analytes after several elimination half-lives, a sensitive and validated method with calibration range of low pg/mL to low ng/mL is needed. Disclosed herein, inter alia, are solutions to these and other problems in the art.

### SUMMARY

The disclosure provides methods for simultaneously detecting and quantifying naloxone, buprenorphine, and norbuprenorphine in biological samples by the steps of (a) obtaining a biological sample using an extraction technique; (b) combining the biological sample with a mobile phase; (c) performing liquid chromatography separation using gradient elution; and (d) determining the concentration of naloxone, buprenorphine, and norbuprenorphine in the biological sample using electrospray ionization mass spectrometry.

The disclosure provides methods for detecting, quantifying, or detecting and quantifying, naloxone, buprenorphine, norbuprenorphine, or a combination of two or three thereof in a biological sample, the methods comprising (a) obtaining a biological sample from a patient, (b) combining the biological sample with a mobile phase to form a first composition; (c) performing liquid chromatography separation on the first composition using gradient elution, thereby forming a second composition; and (d) performing electrospray ionization mass spectrometry on the second composition to detect, quantify, or detect and quantify the naloxone, buprenorphine, norbuprenorphine, or a combination of two or three thereof.

### DESCRIPTION OF THE DRAWINGS

FIGS. 1A-1C. Representative chromatograms of buprenorphine obtained from (FIG. 1A) blank human plasma without spiking IS; (FIG. 1B) plasma spiked with buprenorphine at LLOQ (20.0 pg/mL) and IS; (FIG. 1C) plasma samples from one subject at 1 hour post dose and the buprenorphine concentration was 1229 pg/mL.

FIGS. 2A-2C. Representative chromatograms of norbuprenorphine obtained from (FIG. 2A) blank human plasma without spiking IS; (FIG. 2B) plasma spiked with Norbuprenorphine at LLOQ (20.0 pg/mL) and IS; (FIG. 2C) plasma samples from one subject at 1 hr post dose and the Norbuprenorphine concentration was 191 pg/mL.

FIGS. 3A-3C. Representative chromatograms of naloxone obtained from (FIG. 3A) blank human plasma without spiking IS; (FIG. 3B) plasma spiked with naloxone at LLOQ (1.00 pg/mL) and IS; (FIG. 3C) plasma samples from one subject at 1 hour post dose and the naloxone concentration was 68.0 pg/mL.

FIG. 4. Mean buprenorphine, norbuprenorphine and naloxone concentrations (+SD) versus time on a semi-log scale (n=43). Note: The mean values below LLOQ were set to missing.

### DETAILED DESCRIPTION

It is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

“Liquid chromatography” and “high pressure liquid chromatography” are techniques used to separate, detect, identify, and quantify each component in a mixture. Liquid chromatography (LC) relies on gravity to allow a mobile phase to pass through a column filled with an adsorbent material. High pressure liquid chromatography (HPLC) relies on a pump to pass a pressured mobile phase through a column filled with an adsorbent material. An HPLC device (HPLC) typically includes at least the following: a column packed with a suitable stationary phase, a pump for forcing a mobile phase through the column under pressure, and a detector for detecting the presence of compounds eluting off of the column. The devices include a means for providing for gradient elution, described herein. Routine methods and apparatus for carrying out LC and HPLC separations are well known in the art, and are described, for example, in J. Chromatography, 192:222-227 (1980), J. Liquid Chromatography 4:661-680 (1981), and J. Chromatography, 249:193-198 (1982), the disclosures of which are incorporated by reference herein in their entirety. All the other accessories necessary for carrying out analysis on an LC or HPLC system are well known in the art and not being discussed separately.

“Mass spectrometry” or “MS” refers to methods of filtering, detecting, and measuring ions based on their mass-to-charge ratio, or “m/z.” In general, one or more molecules of interest are ionized, and the ions are subsequently introduced into a mass spectrographic instrument where, due to a combination of magnetic and electric fields, the ions follow a path in space that is dependent upon mass (“m”) and charge (“z”).

Following a liquid chromatography separation, the sample is conveniently detected and quantified by mass spectrometry (MS). The effluent from the LC/HPLC is injected into an ionization chamber of the MS in which a first (parent) ion is produced. The parent ion may be detected directly in a first MS, or it may be isolated by the first MS, fragmented into characteristic daughter ions, and one or more of the daughter ions detected in a second MS (i.e., tandem MS).

“Tandem mass spectrometry,” or “MS/MS” is employed to enhance the resolution of the MS technique. In tandem mass spectrometry, a parent ion generated from a molecule of interest may be filtered in an MS instrument, and the parent ion subsequently fragmented to yield one or more daughter ions that are then analyzed (detected and/or quantified) in a second MS procedure.

“Electrospray ionization” or “ESI” refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber which is heated to prevent condensation and to evaporate solvent. As the droplets get smaller the electrical surface charge density increases until such time that the natural repulsion between like charges causes ions as well as neutral molecules to be released.

“Ionization” refers to the process of generating an analyte ion having a net electrical charge equal to one or more electron units. Negative ions are those having a net negative charge of one or more electron units, while positive ions are those having a net positive charge of one or more electron units. The ions may be detected using several detection modes. For example, selected ions may be detected using a selective ion monitoring mode (SIM) which includes multiple reaction monitoring (MRM) or selected reaction monitoring (SRM). Alternatively, ions may be detected using a scanning mode.

“Gradient elution” is a technique used in liquid chromatographic (LC or HPLC) methods wherein the composition and/or the flow rate of the mobile phase is varied over the course of the experiment. In embodiments, the composition of the mobile phase is varied from low to high eluting strength. In embodiments, the composition of the mobile phase is varied from high to low eluting strength. In embodiments, the composition of the mobile phase is varied according to a set schedule. In embodiments, gradient elution is used to modify the polarity and/or the surface tension of the aqueous mobile phase during the course of the experiment. In embodiments, gradient elution may gradually change the composition of the mobile phase over the course of the experiment. In embodiments, gradient elution may suddenly change the composition of the mobile phase over the course of the experiment. For example there may be two components or the mobile phase, termed “A” and “B.” In embodiments, solvent A is water, salt solution, or an organic solvent (e.g. acetonitrile, methanol, THF, or isopropanol). In embodiments, solvent B is water, salt solution, or an organic solvent (e.g. acetonitrile, methanol, ethanol, tetrahydrofuran, or isopropanol). In embodiments, the pH of the mobile phase is varied over the course of the experiment. In embodiments, solvent A may be a salt in water. In embodiments, solvent B may be a salt in water. In embodiments, solvent A may be a salt in methanol. In embodiments, solvent B may be a salt in methanol. In embodiments, the gradient elution has a schedule shown in Table A. In embodiments, the gradient elution has a schedule shown in Table B. In embodiments, the gradient elution has a schedule shown in Table 1.

TABLE A

| Time | Mobile Phase A | Mobile Phase B | Flow Rate |
|---|---|---|---|
| (min) | (%) | (%) | (mL/min) |
| 0.00 | 25-75 | 25-75 | 0.1 to 1.0 |
| 0.3 to 1.5 | 25-75 | 50 | 0.1 to 1.0 |
| 1 to 3 | 5-40 | 60-95 | 0.1 to 1.0 |
| 2 to 4 | 5-40 | 60-95 | 0.1 to 1.0 |
| 2 to 4 | 5-40 | 60-95 | 0.8 |
| 3 to 5 | 5-40 | 60-95 | 0.8 |
| 4 to 6 | 0-25 | 75-100 | 0.8 |
| 5 to 7 | 0-25 | 75-100 | 0.8 |
| 5 to 7 | 25-75 | 25-75 | 0.8 |
| 5.5 to 8 | 25-75 | 25-75 | 0.8 |
| 5.5 to 10 | 25-75 | 25-75 | 0.1 to 1.0 |

TABLE B

| Time | Mobile Phase A | Mobile Phase B | Flow Rate |
|---|---|---|---|
| (min) | (%) | (%) | (mL/min) |
| 0.00 | 40-60 | 40-60 | 0.4 to 0.6 |
| 0.4 to 1 | 40-60 | 40-60 | 0.4 to 0.6 |
| 1 to 3 | 10-30 | 70-90 | 0.4 to 0.6 |
| 2.5 to 3.5 | 10-30 | 70-90 | 0.4 to 0.6 |
| 2.6 to 4 | 10-30 | 70-90 | 0.6 to 1.0 |
| 3.5 to 4.5 | 10-30 | 70-90 | 0.6 to 1.0 |
| 4 to 5 | 0-20 | 80-100 | 0.6 to 1.0 |
| 5 to 6 | 0-20 | 80-100 | 0.6 to 1.0 |
| 5.1 to 6.5 | 40-60 | 40-60 | 0.6 to 1.0 |
| 5.5 to 7 | 40-60 | 40-60 | 0.6 to 1.0 |
| 5.6 to 8 | 40-60 | 40-60 | 0.4 to 0.6 |

TABLE 1

| Time | Mobile Phase A | Mobile Phase B | Flow Rate |
|---|---|---|---|
| (min) | (%) | (%) | (mL/min) |
| 0.00 | 50 | 50 | 0.5 |
| 0.50 | 50 | 50 | 0.5 |
| 2.00 | 20 | 80 | 0.5 |
| 3.00 | 20 | 80 | 0.5 |
| 3.01 | 20 | 80 | 0.8 |
| 4.00 | 20 | 80 | 0.8 |
| 4.50 | 0 | 100 | 0.8 |
| 5.50 | 0 | 100 | 0.8 |
| 5.51 | 50 | 50 | 0.8 |
| 6.00 | 50 | 50 | 0.8 |
| 6.01 | 50 | 50 | 0.5 |

“Liquid-liquid extraction”, or “solvent extraction” and “partitioning” is a method of separating compounds based on their relative solubilities in two or more different immiscible liquids (e.g., water and an organic solvent). In liquid-liquid extraction, the solutes (e.g., buprenorphine, norbuprenorphine, naloxone) contained in one solution are transferred to another solvent, which is called the extract.

“Solid-phase extraction” is a sample preparation process by which compounds that are dissolved or suspended in a liquid mixture are separated from other compounds in the mixture according to their physical and chemical properties. For example, separating solutes dissolved or suspended in a liquid (e.g. mobile phase) by can be achieved by passing the liquid through another material (e.g. stationary phase) under controlled conditions.

“LLOQ” means lower limit of quantification. In the methods described herein, the LLOQ for naloxone ranges from about 1 pg/ml to about 0.5 pg/ml; the LLOQ for buprenorphine ranges from about 20 pg/ml to about 15 pg/ml; and the LLOQ for norbuprenorphine ranges from about 20 pg/ml to about 15 pg/ml. In embodiments for the quantification of naloxone, buprenorphine, and norbuprenorphine, the method described herein quantifies naloxone in an amount of about 1 pg/ml or more; buprenorphine in an amount of about 20 pg/ml or more; and norbuprenorphine in an amount of about 20 pg/ml or more. In embodiments for the quantification of naloxone, buprenorphine, and norbuprenorphine, the method described herein quantifies naloxone in an amount of about 0.5 pg/ml or more; buprenorphine in an amount of about 15 pg/ml or more; and norbuprenorphine in an amount of about 15 pg/ml or more.

“IS” or “internal standard” refers to a chemical substance added in known quantities to samples for the use in analytical chemistry techniques (e.g. calibration standards). In embodiments, the IS is radiolabeled. In embodiments, the IS comprises deuterated compounds. In embodiments, the IS is Buprenorphine-D₄, Norbuprenorphine-D₃, and Naloxone-D₅.

“CV” or refers to the coefficient of variation and is a standardized measure of dispersion of a probability distribution or frequency distribution. In embodiments, the CV is the ratio of the standard deviation to the mean.

### EMBODIMENTS

In the following embodiments, a reference to a single embodiment is a reference to all the sub-embodiments therein. For example, Embodiment 26 refers to Embodiments 1-25, such that the reference to Embodiment 1 includes references to Embodiment 1A and Embodiment 1B; the reference to Embodiment 2 includes references to Embodiment 2A and Embodiment 2B; and so on.

### Embodiment 1A

A method for quantifying naloxone, buprenorphine, and norbuprenorphine in a human plasma sample comprising: (a) obtaining about 0.1 ml to about 1.0 ml of a plasma sample from a human; (b) performing a liquid-liquid extraction technique on the plasma sample to produce a first composition comprising naloxone, buprenorphine, and norbuprenorphine; (c) combining the first composition with a mobile phase comprising ammonium bicarbonate and methanol to produce a second composition comprising ammonium bicarbonate, methanol, naloxone, buprenorphine, and norbuprenorphine; (d) performing liquid chromatography separation on the second composition using gradient elution to produce a third composition comprising naloxone, buprenorphine, and norbuprenorphine; and (e) performing electrospray ionization mass spectrometry on the third composition to determine the concentration of the buprenorphine, naloxone, and norbuprenorphine; thereby quantifying naloxone, buprenorphine, and norbuprenorphine in a human plasma sample. In embodiments, the naloxone, buprenorphine, and norbuprenorphine are quantified simultaneously.

### Embodiment 1B

A method for quantifying naloxone, buprenorphine, and norbuprenorphine in a human plasma sample comprising: (i) obtaining about 0.1 ml to about 1.0 ml of a plasma sample from a human; (ii) preparing and purifying the plasma sample using a liquid-liquid extraction technique; (iii) combining the plasma sample with a mobile phase comprising ammonium bicarbonate and methanol; (iv) performing liquid chromatography separation on the biological plasma sample using gradient elution; and (v) determining the concentration of the buprenorphine, naloxone, and norbuprenorphine in the plasma sample using electrospray ionization mass spectrometry; thereby quantifying naloxone, buprenorphine, and norbuprenorphine in the human plasma sample. In embodiments, the naloxone, buprenorphine, and norbuprenorphine are quantified simultaneously.

### Embodiment 2A

A method for quantifying naloxone, buprenorphine, and norbuprenorphine in a biological sample comprising: (a) obtaining the biological sample; (b) performing an extraction technique on the biological sample to produce a first composition comprising naloxone, buprenorphine, and norbuprenorphine; (c) combining the first composition with a mobile phase comprising an alcohol to produce a second composition comprising the alcohol, naloxone, buprenorphine, and norbuprenorphine; (d) performing liquid chromatography separation on the second composition using gradient elution to produce a third composition comprising naloxone, buprenorphine, and norbuprenorphine; and (e) performing electrospray ionization mass spectrometry on the third composition to determine the concentration of the buprenorphine, naloxone, and norbuprenorphine; thereby quantifying naloxone, buprenorphine, and norbuprenorphine in the biological sample. In embodiments, the naloxone, buprenorphine, and norbuprenorphine are quantified simultaneously.

### Embodiment 2B

A method for quantifying naloxone, buprenorphine, and norbuprenorphine in a biological sample comprising: (i) obtaining the biological sample from a patient using an extraction technique; (ii) combining the biological sample with a mobile phase comprising an alcohol; (iii) performing liquid chromatography separation on the biological sample using gradient elution; and (iv) determining the concentration of the buprenorphine, naloxone, and norbuprenorphine in the biological sample using electrospray ionization mass spectrometry; thereby quantifying naloxone, buprenorphine, and norbuprenorphine in the biological sample. In embodiments, the naloxone, buprenorphine, and norbuprenorphine are quantified simultaneously.

### Embodiment 3

A method of embodiment of 1 or 2, wherein the electrospray ionization mass is tandem mass spectrometry.

### Embodiment 4

A method of embodiment of 2 or 3, wherein the biological sample is a human biological sample.

### Embodiment 5

A method of any one of embodiments 2, 3, or 4, wherein the biological sample is human serum, human plasma, human tissue, or human cells.

### Embodiment 6

A method of any one of embodiments 2, 3, 4, or 5, wherein the biological sample is human plasma.

### Embodiment 7

A method of any one of embodiments 2, 3, 4, 5, or 6, wherein the biological sample is in an amount of about 0.1 ml to about 1 ml.

### Embodiment 8

A method of any one of embodiments 1-7, wherein the biological sample is in an amount of about 0.1 ml to about 0.5 ml.

### Embodiment 9

A method of any one of embodiments 2-8, wherein the patient is a human.

### Embodiment 10A

A method of any one of embodiments 1-9, wherein the mobile phase has a varied flow rate.

### Embodiment 10B

A method of any one of embodiments 1-9, wherein the mobile phase has a flow rate from about 0.1 mL/min to about 1.5 mL/min.

### Embodiment 10C

A method of any one of embodiments 1-9, wherein the mobile phase has a flow rate from about 0.2 mL/min to about 1.2 mL/min.

### Embodiment 10D

A method of any one of embodiments 1-9, wherein the mobile phase has a flow rate from about 0.3 mL/min to about 1.0 mL/min.

### Embodiment 10E

A method of any one of embodiments 1-9, wherein the mobile phase has a flow rate from about 0.4 mL/min to about 0.9 mL/min.

### Embodiment 10F

A method of any one of embodiments 1-9, wherein the mobile phase has a flow rate from about 0.5 mL/min to about 0.8 mL/min.

### Embodiment 11A

A method of any one of embodiments 1-10, wherein the gradient elution has a schedule shown in Table 1.

### Embodiment 11B

A method of any one of embodiments 1-10, wherein the gradient elution has a schedule shown in Table 1; wherein Mobile Phase A comprises ammonium bicarbonate in water; and wherein Mobile Phase B comprises ammonium bicarbonate in methanol.

### Embodiment 11C

A method of any one of embodiments 1-10, wherein the gradient elution has a schedule shown in Table 1; wherein Mobile Phase A comprises about 0.5 mM to about 5 mM ammonium bicarbonate in water; and wherein Mobile Phase B comprises about 1 mM to about 10 mM ammonium bicarbonate in methanol.

### Embodiment 11D

A method of any one of embodiments 1-10, wherein the gradient elution has a schedule shown in Table 1; wherein Mobile Phase A comprises about 1 mM to about 3 mM ammonium bicarbonate in water; and wherein Mobile Phase B comprises about 4 mM to about 6 mM ammonium bicarbonate in methanol.

### Embodiment 11E

A method of any one of embodiments 1-10, wherein the gradient elution has a schedule shown in Table 1; wherein Mobile Phase A comprises about 1.5 mM to about 2.5 mM ammonium bicarbonate in water; and wherein Mobile Phase B comprises about 4.5 mM to about 5.5 mM ammonium bicarbonate in methanol.

### Embodiment 11F

A method of any one of embodiments 1-10, wherein the gradient elution has a schedule shown in Table 1; wherein Mobile Phase A comprises about 2 mM ammonium bicarbonate in water; and wherein Mobile Phase B comprises about 5 mM ammonium bicarbonate in methanol.

### Embodiment 11G

A method of any one of embodiments 1-10, wherein the gradient elution has a schedule shown in Table A or Table B.

### Embodiment 11H

A method of any one of embodiments 1-10, wherein the gradient elution has a schedule shown in Table A or Table B; wherein Mobile Phase A comprises ammonium bicarbonate in water; and wherein Mobile Phase B comprises ammonium bicarbonate in methanol.

### Embodiment 11I

A method of any one of embodiments 1-10, wherein the gradient elution has a schedule shown in Table A or Table B; wherein Mobile Phase A comprises about 0.5 mM to about 5 mM ammonium bicarbonate in water; and wherein Mobile Phase B comprises about 1 mM to about 10 mM ammonium bicarbonate in methanol.

### Embodiment 11J

A method of any one of embodiments 1-10, wherein the gradient elution has a schedule shown in Table A or Table B; wherein Mobile Phase A comprises about 1 mM to about 3 mM ammonium bicarbonate in water; and wherein Mobile Phase B comprises about 4 mM to about 6 mM ammonium bicarbonate in methanol.

### Embodiment 11K

A method of any one of embodiments 1-10, wherein the gradient elution has a schedule shown in Table A or Table B; wherein Mobile Phase A comprises about 1.5 mM to about 2.5 mM ammonium bicarbonate in water; and wherein Mobile Phase B comprises about 4.5 mM to about 5.5 mM ammonium bicarbonate in methanol.

### Embodiment 11L

A method of any one of embodiments 1-10, wherein the gradient elution has a schedule shown in Table A or Table B; wherein Mobile Phase A comprises about 2 mM ammonium bicarbonate in water; and wherein Mobile Phase B comprises about 5 mM ammonium bicarbonate in methanol.

### Embodiment 12A

A method of any one of embodiments 1-11, wherein the gradient elution has a schedule and flow rate shown in Table 1.

### Embodiment 12B

A method of any one of embodiments 1-11, wherein the gradient elution has a schedule and flow rate shown in Table 1; wherein Mobile Phase A comprises ammonium bicarbonate in water; and wherein Mobile Phase B comprises ammonium bicarbonate in methanol.

### Embodiment 12C

A method of any one of embodiments 1-11, wherein the gradient elution has a schedule and flow rate shown in Table 1; wherein Mobile Phase A comprises about 0.5 mM to about 5 mM ammonium bicarbonate in water; and wherein Mobile Phase B comprises about 1 mM to about 10 mM ammonium bicarbonate in methanol.

### Embodiment 12D

A method of any one of embodiments 1-11, wherein the gradient elution has a schedule and flow rate shown in Table 1; wherein Mobile Phase A comprises about 1 mM to about 3 mM ammonium bicarbonate in water; and wherein Mobile Phase B comprises about 4 mM to about 6 mM ammonium bicarbonate in methanol.

### Embodiment 12E

A method of any one of embodiments 1-11, wherein the gradient elution has a schedule and flow rate shown in Table 1; wherein Mobile Phase A comprises about 1.5 mM to about 2.5 mM ammonium bicarbonate in water; and wherein Mobile Phase B comprises about 4.5 mM to about 5.5 mM ammonium bicarbonate in methanol.

### Embodiment 12F

A method of any one of embodiments 1-11, wherein the gradient elution has a schedule and flow rate shown in Table 1; wherein Mobile Phase A comprises about 2 mM ammonium bicarbonate in water; and wherein Mobile Phase B comprises about 5 mM ammonium bicarbonate in methanol.

### Embodiment 12G

A method of any one of embodiments 1-11, wherein the gradient elution has a schedule and flow rate shown in Table A or Table B.

### Embodiment 12H

A method of any one of embodiments 1-11, wherein the gradient elution has a schedule and flow rate shown in Table A or Table B; wherein Mobile Phase A comprises ammonium bicarbonate in water; and wherein Mobile Phase B comprises ammonium bicarbonate in methanol.

### Embodiment 12I

A method of any one of embodiments 1-11, wherein the gradient elution has a schedule and flow rate shown in Table A or Table B; wherein Mobile Phase A comprises about 0.5 mM to about 5 mM ammonium bicarbonate in water; and wherein Mobile Phase B comprises about 1 mM to about 10 mM ammonium bicarbonate in methanol.

### Embodiment 12J

A method of any one of embodiments 1-11, wherein the gradient elution has a schedule and flow rate shown in Table A or Table B; wherein Mobile Phase A comprises about 1 mM to about 3 mM ammonium bicarbonate in water; and wherein Mobile Phase B comprises about 4 mM to about 6 mM ammonium bicarbonate in methanol.

### Embodiment 12K

A method of any one of embodiments 1-11, wherein the gradient elution has a schedule and flow rate shown in Table A or Table B; wherein Mobile Phase A comprises about 1.5 mM to about 2.5 mM ammonium bicarbonate in water; and wherein Mobile Phase B comprises about 4.5 mM to about 5.5 mM ammonium bicarbonate in methanol.

### Embodiment 12L

A method of any one of embodiments 1-11, wherein the gradient elution has a schedule and flow rate shown in Table A or Table B; wherein Mobile Phase A comprises about 2 mM ammonium bicarbonate in water; and wherein Mobile Phase B comprises about 5 mM ammonium bicarbonate in methanol.

### Embodiment 13

A method of any one of embodiments 2-12, wherein the extraction technique is a solid-phase extraction technique.

### Embodiment 14

A method of any one of embodiments 2-12, wherein the extraction technique is a liquid-liquid extraction technique.

### Embodiment 15A

A method of any one of embodiments 1-12 and 14, wherein the liquid-liquid extraction comprises a C₂₋₆ linear or branched alkyl ether and a C₁₋₁₀ linear or branched alkane. In embodiments, the liquid-liquid extraction comprises a C₄₋₆ linear or branched alkyl ether and a C₅₋₇ linear or branched alkane. In embodiments, the liquid-liquid extraction comprises a C₅ linear or branched alkyl ether and a C₆ linear or branched alkane. In embodiments, the ether and the alkane are present in a volume ration (v:v) of 3:1 to 1:1.

### Embodiment 15B

A method of any one of embodiments 1-12 and 14, wherein the liquid-liquid extraction comprises methyl tert-butyl ether and hexane.

### Embodiment 15C

A method of any one of embodiments 1-12 and 14, wherein the liquid-liquid extraction comprises methyl tert-butyl ether and hexane in a volume ratio (v:v) of 3:1 to 1:1.

### Embodiment 15D

A method of any one of embodiments 1-12 and 14, wherein the liquid-liquid extraction technique comprises methyl tert-butyl ether:hexane in a volume ratio (v:v) of 2:1.

### Embodiment 16

A method of any one of embodiments 1-15, wherein the mobile phase is alkaline.

### Embodiment 17

A method of any one of embodiments 2-16, wherein the mobile phase comprises ammonium bicarbonate.

### Embodiment 18

A method of any one of embodiments 2-17, wherein the mobile phase comprises ammonium hydroxide.

### Embodiment 19A

A method of any one of embodiments 1-10 and 13-18, wherein the mobile phase comprises ammonium bicarbonate in water.

### Embodiment 19B

A method of any one of embodiments 1-10 and 13-18, wherein the mobile phase comprises about 0.5 mM to about 5 mM ammonium bicarbonate in water.

### Embodiment 19C

A method of any one of embodiments 1-10 and 13-18, wherein the mobile phase comprises about 1 mM to about 3 mM ammonium bicarbonate in water.

### Embodiment 19D

A method of any one of embodiments 1-10 and 13-18, wherein the mobile phase comprises about 1.5 mM to about 2.5 mM ammonium bicarbonate in water.

### Embodiment 19E

A method of any one of embodiments 1-10 and 13-18, wherein the mobile phase comprises about 2 mM ammonium bicarbonate in water.

### Embodiment 20A

A method of any one of embodiments 1-10 and 13-19, wherein the mobile phase comprises ammonium bicarbonate in methanol.

### Embodiment 20B

A method of any one of embodiments 1-10 and 13-19, wherein the mobile phase comprises about 1 mM to about 10 mM ammonium bicarbonate in methanol.

### Embodiment 20C

A method of any one of embodiments 1-10 and 13-19, wherein the mobile phase comprises about 4 mM to about 6 mM ammonium bicarbonate in methanol.

### Embodiment 20D

A method of any one of embodiments 1-10 and 13-19, wherein the mobile phase comprises about 4.5 mM to about 5.5 mM ammonium bicarbonate in methanol.

### Embodiment 20E

A method of any one of embodiments 1-10 and 13-19, wherein the mobile phase comprises about 5 mM ammonium bicarbonate in methanol.

### Embodiment 21

A method of any one of embodiments 1-20, wherein the mobile phase has a pH of about 5 to about 10.

### Embodiment 22

A method of any one of embodiments 1-20, wherein the mobile phase has a pH of about 6 to about 9.

### Embodiment 23

A method of any one of embodiments 1-20, wherein the mobile phase has a pH of about 7.5 to about 8.5.

### Embodiment 24

A method of any one of embodiments 1-20, wherein the mobile phase has a pH of about 7.8 to about 8.0.

### Embodiment 25

A method of any one of embodiments 1-20, wherein the mobile phase has a pH of about 7.9.

### Embodiment 26

A method of any one of embodiments 1-25, wherein the liquid chromatography is high pressure liquid chromatography.

### EXAMPLES

The following examples serve to illustrate the invention. These examples are in no way intended to limit the scope of the invention.

Chemicals and reagents. Separate reference solutions containing 100 μg/mL of buprenorphine, 100 μg/mL of norbuprenorphine, and 1.0 mg/mL of naloxone were obtained from Cerilliant (Round Rock, Tex., USA). Buprenorphine-D₄ (100 μg/mL), Norbuprenorphine-D₃ (100 μg/mL) and Naloxone-D₅ (100 μg/mL), used as internal standards (IS), were also obtained from Cerilliant. HPLC-grade methanol, hexane, Methyl t-Butyl Ether (MTBE), and 2-propanol were purchased from EMD Millipore (Billerica, Mass., USA). Reagent grade ammonium bicarbonate, ammonium hydroxide (28.0 to 30.0 w/w %) and trifluoroacetic acid were purchased from Fisher Scientific (Pittsburgh, Pa., USA). Water was purified using Barnstead water purification system (Thermo Scientific, Waltham, Mass.). Human plasma (K₂ EDTA) was obtained from Valley Biomedical (Winchester, Va., USA) and was used as blank matrix for the preparation of calibration standards, quality control samples (QCs) and blank samples.

Instrumentation. A Shimadzu HPLC system, consisting of a SIL-20AC autosampler, LC-20AD binary pump, DGU-20A5R degassing unit, and CTO-20AC thermostatted column oven, was used in this study (Shimadzu, Kyoto, Japan). The mass spectrometer utilized for this work was a Sciex API 5500 triple-quadrupole mass spectrometer equipped with a Turboion Spray source (AB Sciex, Foster City Calif., USA). Study data were collected using Analyst® (Version 1.5.1, Applied Biosystems/MDS Sciex) and evaluated with Watson Laboratory Information Management System™ (LIMS; version 7.4.1, Thermo Fisher Scientific) software.

Liquid chromatographic and mass spectrometric conditions. Chromatographic separation was performed on a Unison UK-C18 column (2.0×50 mm; 3 μm) (Imtakt, Portland, Oreg., USA). Mobile phase A consisted of 2 mM ammonium bicarbonate in water (pH=7.9) and mobile phase B consisted of 5 mM ammonium bicarbonate in methanol. A 10-μL injection of each sample was loaded onto the column, separated and eluted using the gradient shown in Table 1 above. The total run time was 6.5 min and the column temperature was maintained at 35° C. The autosampler injection needle was washed with methanol:water:trifluoroacetic acid (50:50:0.2, v:v:v) to reduce carry-over after each injection. The mass spectrometer was run in positive ion ESI mode using multiple-reaction monitoring (MRM) to monitor the mass transitions. The ion spray voltage and the source temperature was set at 4000 V and 550° C., respectively. Nitrogen gas was used as the curtain gas (set at 30.0) and the collisionally activated dissociation (CAD) gas (set at 10.0). The ion source gas 1 was set at 80.0 and ion source gas 2 was set at 65.0. The resolutions for both Q1 and Q3 were set at unit. A summary of the ion transitions, declustering potentials, collision energies, and collision cell exit potentials are presented in Table 2.

TABLE 2
Optimal positive ion ESI mass spectrometric
conditions for multiple reaction monitoring.

| | | | | | Collision |
|---|---|---|---|---|---|
| | | Dwell | Declustering | Collision | Cell Exit |
| | | time | Potential | Energy | Potential |
| Drug | Ion Transition | (ms) | (V) | (V) | (V) |
| buprenorphine | 468.5→414.4 | 50 | 100 | 55 | 8 |
| buprenorphine-D₄ (IS) | 472.5→414.4 | 50 | 100 | 48 | 8 |
| norbuprenorphine | 414.4→340.3 | 50 | 100 | 42 | 8 |
| Norbuprenorphine-D₃ (IS) | 417.4→343.3 | 50 | 100 | 42 | 8 |
| naloxone | 328.2→253.2 | 50 | 100 | 37 | 8 |
| naloxone-D₅ (IS) | 333.2→212.2 | 50 | 100 | 52 | 8 |

Preparation of stock, calibration standards and quality control samples. Combined stock solution of 1 μg/mL of buprenorphine and norbuprenorphine was prepared by diluting the 100 μg/mL buprenorphine and 100 μg/mL norbuprenorphine reference standard solutions with methanol:water (50:50, v:v). Stock solution of 100 ng/mL of naloxone was prepared by diluting the 1.0 mg/mL naloxone reference standard solution with methanol:water (50:50, v:v). Combined working standard solutions with concentrations of 100/100/5 ng/mL and 80/80/4 ng/mL for buprenorphine/norbuprenorphine/naloxone were prepared by dilution of stock solution of buprenorphine and norbuprenorphine at 1 μg/m L and stock solution of naloxone at 100 ng/mL with methanol:water (50:50, v:v). Additional working standard solutions for buprenorphine/norbuprenorphine/naloxone with concentrations of 0.2/0.2/0.01, 0.4/0.4/0.02, 1/1/0.05, 3/3/0.15, 10/10/0.5 and 30/30/1.5 ng/mL were prepared from 100/100/5 ng/mL and 80/80/4 ng/mL buprenorphine/norbuprenorphine/naloxone standard solutions. A combined working internal standard solution with buprenorphine-d₄ (5 ng/mL), norbuprenorphine-d₃ (5 ng/mL) and naloxone-d₅ (0.25 ng/mL) was prepared by dilution of 100 μg/mL buprenorphine-d₄, 100 μg/mL norbuprenorphine-d₃ and 100 μg/mL and naloxone-d₅ standard solutions with methanol:water (50:50, v:v). Stock solutions, working standard solutions and working internal standard solutions were kept at −20° C. Calibration standard plasma samples for buprenorphine/norbuprenorphine/naloxone (20/20/1, 40/40/1, 100/100/5, 300/300/15, 1000/1000/50, 3000/3000/150, 8000/8000/400, 10000/10000/500 pg/mL) were freshly prepared by spiking 50 μL of working standard solutions into 500 μL of blank human K₂ EDTA plasma on each day of validation and sample analysis. QC samples with concentrations of 60, 800, 7500 and 15000 (dilution QC) pg/mL for buprenorphine and norbuprenorphine, and 3, 40, 375 and 750 (dilution QC) pg/mL for naloxone were prepared in ice-water bath by dilution of a second stock solution of buprenorphine and norbuprenorphine at 1 μg/mL and 100 ng/mL of naloxone stock solution with blank human K₂ EDTA plasma. All of the QC samples were stored at approximately −20° C.

Sample preparation. To a 500 μL of plasma sample, 50 μL of methanol:water (50:50, v:v) and 50 μL combined internal standard solution (5/5/0.25 ng/mL for buprenorphine-d₄/norbuprenorphine-d₃/naloxone-d₅) were spiked and the sample shaken for 5 seconds. After adding 50 μL of 1M ammonium hydroxide, the samples were briefly mixed and extracted in 2.0 mL MTBE:hexane (2:1, v:v) for approximately 30 seconds. The samples were then centrifuged at 1100×g at 10° C. for 5 min. The samples were frozen in the dry-ice bath and then the upper organic layer was removed and evaporated to dryness at 40° C. under a gentle stream of nitrogen (9 psi). The dried extracts were reconstituted in 0.1 mL of methanol:water (50:50, v:v) and were kept refrigerated until analysis by LC-MS/MS.

The analytical procedure was validated for linearity, recovery, matrix effect, accuracy and precision according to the Food and Drug Administration (FDA) and European Medicines Agency (EMA) guidance. Plasma calibration curves were constructed using the peak area ratios of buprenorphine, norbuprenorphine and naloxone to their isotope labeled IS, and applying separate weighted (1/x²) least squares linear regression analyses. Precision (expressed as % coefficient of variation, CV) and accuracy (expressed as % bias) were calculated for four QC samples (LLOQ, low, medium and high). Six replicates of each QC point were analyzed to determine the intra-day accuracy and precision. This process was repeated three times over three different days in order to determine the inter-day accuracy and precision.

Recovery was evaluated in six replicates, at low, medium and high levels. The instrument response of pre-extraction spiked samples prepared in blank matrix (prepared by addition of analyte and IS to blank matrix prior to extraction) was compared to the instrument response of post-extraction spiked samples prepared in extracted blank matrix (prepared by addition of analyte and IS to blank matrix extract). Matrix effect (reported as matrix factor) was evaluated in six different lots (individual, non-pooled), in singlicate, at high and low levels. The instrument response of post-extraction spiked samples prepared in extracted blank matrix (prepared by addition of analyte and IS to blank matrix extract) was compared to the instrument response of matrix-free post-spiked samples (prepared in the absence of blank matrix). In addition, hemolysis effect (plasma containing 2.5% hemolyzed human whole blood) and lipemic plasma effect (approximately 3 mg/mL triglyceride in plasma) were also evaluated in six replicates, at two levels of buprenorphine/norbuprenorphine/naloxone QC samples (60/60/3 and 7500/7500/375 pg/mL).

Stock solution stability was determined at storage conditions of −20° C. for 610 days and at room temperature for 8.5 h. Analytes were considered stable if the relative percent difference between the control and stressed solutions was within ±10.0%. Analyte stability was evaluated in matrix and sample extracts at low and high QC levels (n=6 for each level). The QC samples were analyzed against a freshly prepared calibration curve and the obtained concentrations were compared to the nominal concentrations. The analytes were considered stable if the mean concentration at each level was within 15% of the nominal concentration and the CV % did not exceed 15%. The bench-top stability of samples stored in an ice-water bath was evaluated for 6.2 h. The freeze/thaw stability was evaluated by comparing stability samples following four freeze/thaw cycles. The extract stability and long-term stability were evaluated after the samples were kept in a refrigerator at 2-8° C. for 73.8 hours and at −20° C. for 606 days, respectively.

Six different (individual, non-pooled) lots of blank plasma were analyzed to identify any interference at the retention time of analytes (buprenorphine, norbuprenorphine and naloxone) or their internal standards. No interfering peaks from endogenous compounds were observed at the retention times of all three analytes and their isotope labeled internal standards.

The selectivity of the method was evaluated by spiking the LLOQ standard into six different lots of blank plasma and extracting to examine the effect of variation in matrix. The values obtained from different lots of plasma should not deviate by more than 20% of the nominal value of the LLOQ. The ranges in bias from six lots of plasma were: −5.8% to 7.8% for buprenorphine, −19.0% to 4.8% for norbuprenorphine and −5.6% to 11.0% for naloxone.

The matrix blank sample was injected following the upper limit of quantitation (ULOQ) standard to assess the carryover effects. The peak responses at the retention time of the analyte and internal standard were ≤20.0% and ≤5%, respectively, of the peak response of the LLOQ standard analyzed in the same run. The results indicated no significant carryover was observed in all runs for both assays during the validation.

Linearity and sensitivity. The peak area ratios (y) of three analytes to the internal standards and the concentrations of the calibration standards (x) were fitted by a weighted linear least squares regression analysis to the equation y=a+bx, where a is the y-intercept and b is the slope of the calibration curve. The weighting of 1/x² was selected based on power for weights calculations. The linearity of the method was demonstrated over the calibration range of 20 to 10000 pg/mL for buprenorphine and norbuprenorphine, and 1 to 500 pg/mL for naloxone. The correlation coefficients (R²) for all of the calibration curves were higher than 0.995 for buprenorphine, 0.997 for norbuprenorphine and 0.996 for naloxone. No significant trend or bias was observed. Table 3 shows the slope, y-intercept and R² values generated from the calibration curves during validation.

TABLE 3
Statistical data for linearity including standard deviation (S.D.) (linear range:
20-10,000 pg/mL for buprenorphine and norbuprenorphine; 1-500 pg/mL for naloxone).

| | buprenorphine | norbuprenorphine | naloxone |
|---|---|---|---|
| | (n = 8) | (n = 8) | (n = 8) |
| R² | 0.9958-0.9989 | 0.9972-0.9995 | 0.9960-0.9996 |
| Slope | 0.00372 ± 0.00026 | 0.00200 ± 0.000070 | 0.0367 ± 0.0012 |
| Intercept | −0.00357 ± 0.0028 | 0.000523 ± 0.0021 | 0.0000210 ± 0.0030 |

The LLOQ, defined as the lowest concentration of analyte with accuracy within 20% and a precision <20%, was 20 pg/mL for buprenorphine and norbuprenorphine, and 1 pg/mL for naloxone as shown in Table 4.

TABLE 4
The intra-day (n = 6) and inter-day (n = 18) precision (% CV) and accuracy (%
bias) of the LC-MS/MS method used to quantitate buprenorphine, norbuprenorphine and
naloxone in human K₂ EDTA plasma.

| | | Intra-day | | | Inter-day | | |
|---|---|---|---|---|---|---|---|
| | Concentration | Observed | | | Observed | | |
| | added | concentration ± | CV | Bias | concentration | CV | Bias |
| Drug | (pg/mL) | S.D. (pg/mL) | (%) | (%) | (pg/mL) | (%) | (%) |
| buprenorphine | 20.0 | 19.9 ± 0.683 | 3.4 | −0.1 | 20.0 ± 0.706 | 3.5 | −0.2 |
| | 60.0 | 59.3 ± 2.15 | 3.6 | −1.2 | 59.4 ± 1.83 | 3.1 | −0.9 |
| | 800 | 732 ± 28.7 | 3.9 | −8.5 | 722 ± 29.3 | 4.1 | −9.8 |
| | 7500 | 7367 ± 266 | 3.6 | −1.8 | 7272 ± 247 | 3.4 | −3.0 |
| | 15000 | 14652 ± 465 | 3.2 | −2.3 | | | |
| Norbuprenorphine | 20.0 | 18.1 ± 1.00 | 5.5 | −9.3 | 19.1 ± 1.58 | 8.3 | −4.7 |
| | 60.0 | 57.9 ± 3.12 | 5.4 | −3.5 | 59.4 ± 2.62 | 4.4 | −0.9 |
| | 800 | 772 ± 34.0 | 4.4 | −3.5 | 768 ± 30.2 | 3.9 | −4.0 |
| | 7500 | 7175 ± 200 | 2.8 | −4.3 | 7237 ± 296 | 4.1 | −3.5 |
| | 15000 | 14049 ± 333 | 2.4 | −6.3 | | | |
| naloxone | 1.00 | 0.969 ± 0.106 | 11.0 | −3.1 | 0.971 ± 0.0923 | 9.5 | −2.9 |
| | 3.00 | 3.03 ± 0.192 | 6.4 | 0.9 | 2.99 ± 0.132 | 4.4 | −0.2 |
| | 40.0 | 38.1 ± 0.895 | 2.4 | −4.8 | 37.6 ± 0.944 | 2.5 | −6.4 |
| | 375 | 363 ± 11.4 | 3.1 | −3.1 | 362 ± 12.1 | 3.3 | −3.5 |
| | 750 | 749 ± 14.0 | 1.9 | −0.2 | | | |

A signal-to-noise (S/N) >5 at the LLOQ for all three analytes was observed. To our knowledge, this is the first time that the LLOQ was as low as 1 pg/mL for naloxone in validated methods for biological samples.

Precision and accuracy were evaluated by analyzing six samples at the LLOQ, low, medium and high QC levels (20, 60, 800, and 7500 pg/mL for buprenorphine and norbuprenorphine; 3, 40, 375, and 750 pg/mL for naloxone). The accuracy and precision data are summarized in Table 4. The intra-day precision (% CV) was ≤11.0%, and the inter-day precision was 9.5% for all three analytes. The intra-day and inter-day accuracy (% bias) were within ±9.8%. Dilution integrity was conducted to evaluate 2-fold dilution. Six replicates of the dilution QC samples were diluted 2× with blank plasma prior to analysis. The intra-day precision and accuracy for dilution QCs (Table 4) were all within 15% for all three analytes.

Recovery and matrix effect. Recovery and matrix factor data for buprenorphine, norbuprenorphine, naloxone and their internal standards are summarized in Table 5 and Table 6.

TABLE 5
Recovery of buprenorphine, norbuprenorphine, naloxone and their
internal standards (mean ± S.D.) from human K₂ EDTA plasma.

| | | Recovery |
|---|---|---|
| | Concentration | (%) |
| Drug | (pg/mL) | n = 6 |
| buprenorphine | 40.0 | 63.1 ± 2.3 |
| | 1000 | 64.7 ± 1.4 |
| | 10000 | 67.3 ± 0.8 |
| buprenorphine-d4 (IS) | 5000 | 67.8 ± 3.7 |
| norbuprenorphine | 40.0 | 83.1 ± 6.8 |
| | 1000 | 77.7 ± 1.6 |
| | 10000 | 80.8 ± 1.4 |
| norbuprenorphine-d3 (IS) | 5000 | 78.2 ± 2.8 |
| naloxone | 2.00 | 78.2 ± 7.2 |
| | 50.0 | 76.2 ± 1.0 |
| | 500 | 77.2 ± 1.6 |
| naloxone-d5 (IS) | 250 | 75.0 ± 3.4 |

TABLE 6
Matrix factor of buprenorphine, norbuprenorphine, naloxone and their internal
standards (mean ± S.D.) from 6 different lots of human K₂ EDTA plasma.

| | Concentration | Matrix | IS Matrix | IS-Normalized Matrix Factor |
|---|---|---|---|---|
| Drug | (pg/mL) | Factor | Factor | (CV %) |
| buprenorphine | 60.0 | 0.744± | 0.677± | 1.10 ± 0.047 (4.3%) |
| | 7500 | 0.794± | 0.592± | 1.33 ± 0.14 (10.2%) |
| norbuprenorphine | 60.0 | 0.882± | 0.919± | 0.961 ± 0.066 |
| | 7500 | 1.05 ± 0.10 | 1.09± | 0.962 ± 0.035 |
| naloxone | 3.00 | 0.985± | 0.963± | 1.02 ± 0.060 (5.9%) |
| | 375 | 1.04± | 1.09± | 0.958 ± 0.025 |

The recoveries ranged from 63.1% to 67.3% for buprenorphine, 77.7% to 83.1% for norbuprenorphine, and 76.2% to 78.2% for naloxone. Recoveries for the isotope labeled internal standards were 67.8%, 78.2% and 75.0%, respectively. The difference between concentration levels and between the analytes and internal standards were within 20%. The recoveries for buprenorphine were slightly lower than norbuprenorphine and naloxone because the extraction conditions were optimized to improve the sensitivity for norbuprenorphine and naloxone. Matrix effects are an important issue in ESI. The matrix factors for norbuprenorphine, naloxone and their IS in six different lots of plasma ranged from 0.882 to 1.09. The IS normalized matrix factors which were calculated by dividing the MF of the analyte by the MF of the IS were around 1.0 and the CV was lower than 6.8%. These results demonstrated that the sample preparation procedure of the LLE method used in this assay provided a clean extraction solution for LC-MS/MS, which essentially reduces the matrix effect. However, the MF of buprenorphine and IS (buprenorphine-D₄) ranged from 0.592 to 0.794, which suggested a slight ion suppression effect. This effect is mainly due to other compounds co-eluting with buprenorphine when the gradient of the mobile phase was changed to high organic. The IS normalized MFs for buprenorphine were around 1.1, which also demonstrated the ion suppression effect was tracked and minimized by the isotope labeled IS. In addition, hemolysis effect and lipemic effect were also evaluated at low and high QC levels. The accuracy and precision for all analytes were within 8.6%.

Stability studies. The stock solutions were stable at −20° C. for 610 days and at room temperature for 8.5 hours. Results of the stability studies in matrix and extract are presented in Table 7.

TABLE 7
Stability testing of buprenorphine, norbuprenorphine and
naloxone extracted from human K₂ EDTA plasma (n = 6).

| | | Spiked | Observed | | |
|---|---|---|---|---|---|
| | | Concentration | Concentration ± | CV | Bias |
| Drug | Stability | (pg/mL) | S.D. (ng/ml) | (%) | (%) |
| BUP | Bench top (6.2 h) | 60.0 | 54.8 ± 2.00 | 3.7 | −8.7 |
| | | 7500 | 6705 ± 88.5 | 1.3 | −10.6 |
| | Freeze-thaw (4 cycles) | 60.0 | 60.2 ± 4.91 | 8.2 | 0.4 |
| | | 7500 | 7346 ± 272 | 3.7 | −2.1 |
| | Extract (74 h) | 60.0 | 60.5 ± 1.78 | 2.9 | 0.9 |
| | | 7500 | 7452 ± 316 | 4.2 | −0.6 |
| | Long-term (−20° C., 606 | 60.0 | 57.6 ± 3.11 | 5.4 | −4.1 |
| | | 7500 | 6924 ± 401 | 5.8 | −7.7 |
| NorBU | Bench top | 60.0 | 58.5 ± 2.82 | 4.8 | −2.5 |
| | (6.2 h) | 7500 | 7110 ± 146 | 2.0 | −5.2 |
| | Freeze-thaw | 60.0 | 59.5 ± 1.46 | 2.5 | −0.8 |
| | (4 cycles) | 7500 | 7608 ± 367 | 4.8 | 1.4 |
| | Extract | 60.0 | 58.2 ± 4.35 | 7.5 | −3.0 |
| | (74 h) | 7500 | 7339 ± 217 | 3.0 | −2.1 |
| | Long-term (−20° C. 606 | 60.0 | 55.6 ± 1.34 | 2.4 | −7.4 |
| | | 7500 | 7110 ± 169 | 2.4 | −5.2 |
| NALna | Bench top (6.2 h) | 3.00 | 2.97 ± 0.0943 | 3.2 | −1.1 |
| | | 375 | 380 ± 14.5 | 3.8 | 1.3 |
| | Freeze-thaw (4 cycles) | 3.00 | 2.85 ± 0.172 | 6.0 | −5.0 |
| | | 375 | 359 ± 14.5 | 4.0 | −4.4 |
| | Extract (74 h) | 3.00 | 2.96 ± 0.105 | 3.5 | −1.2 |
| | | 375 | 397 ± 25.9 | 6.5 | 6.0 |
| | Long-term (−20° C., 606 | 3.00 | 2.82 ± 0.257 | 9.1 | −6.0 |
| | | 375 | 339 ± 5.24 | 1.5 | −9.6 |

Abbreviations: BUP=buprenorphine; NAL=naloxone; NorBUP=norbuprenorphine

The bias % ranged from −10.6% to 6.0% (<15%) and CV ranged from 1.3% to 9.1% for all three analytes. Stability results indicate that buprenorphine, norbuprenorphine and naloxone were stable in unextracted human plasma on the benchtop for 6.2 hours in an ice-water bath, after four freeze/thaw cycles; in the extracted solution when stored under refrigerated conditions (2-8° C.) for at least 73.8 hours and in human plasma samples stored at −20° C. for at least 606 days. Reinjection reproducibility testing was also performed by re-injecting a previously accepted run to determine if an analytical run can be reanalyzed. In the re-injected runs for all three analytes, all QC samples (LLOQ, low, medium and high QCs) were within 15% of their theoretical concentrations.

The developed and validated LC-MS/MS assay was used to investigate the pharmacokinetic profiles of buprenorphine, norbuprenorphine and naloxone after a single dose of Suboxone sublingual tablet (buprenorphine/naloxone 2 mg/0.5 mg) in healthy subjects. The clinical study protocol, informed consent form(s), and all other study-related documents were reviewed and approved by an independent and appropriately constituted institutional review board. All subjects provided informed consent before they enrolled in the study. To mitigate potential adverse effects associated with buprenorphine administration, naltrexone was administered at 12 and 1 hours pre-dose, in addition to 24 hours post-Suboxone administration. Series blood samples were collected pre-dose, and at 0.083, 0.25, 0.5, 0.75, 1, 1.25, 1.5, 2, 3, 4, 6, 8, 10, 12, 24, 36, 48, 72, 96, 120, and 144 hours post-dose. Pharmacokinetic analysis was performed using a non-compartmental method by WinNonlin (version 6.3; Pharsight Corporation, Mountain View, Calif., USA).

Following sublingual administration, naloxone dose is only 25% of that of buprenorphine. In addition, when taken sublingually, naloxone has poorer absorption (40% for buprenorphine vs. 10% for naloxone for the solution formulation) and a shorter duration of action (1 day for buprenorphine vs. 1 hour for naloxone) compared to buprenorphine. Therefore, the naloxone LLOQ is targeted at 20 times lower than buprenorphine and norbuprenorphine. Chromatographic conditions were optimized to achieve maximum sensitivity of naloxone. Unison UK-C18 column from Imtakt gave the sharpest peak of naloxone, while buprenorphine and norbuprenorphine also had satisfactory performance. Larger particle size column (3 μm) from this supplier was selected since it exhibited similar performance as sub −2 μm columns from other suppliers but showed lower operating pressure. Alkaline mobile phase (ammonium bicarbonate in aqueous and organic phase) was used to improve the retention of naloxone. Naloxone was therefore eluted in a mobile phase having higher organic content, which dramatically increased LC-MS sensitivity. Ammonium bicarbonate buffers are more likely to change pH over time at room temperature, because they will slowly decompose to ammonia and carbonate in the buffer, and subsequently be lost due to evaporation. The pH of mobile phases can affect the retention time, separation and peak shapes of the analytes. To keep the pH values of the mobile phases with ammonium bicarbonate buffer stable, the mobile phases were kept in a thermoelectric cooler at approximately 1° C. during running. These mobile phases stored at the above conditions can be used for two days before being replaced by freshly prepared ones.

This clinical study was conducted in healthy volunteers using naltrexone, an opioid antagonist, to minimize the occurrence of unacceptable adverse effects that may be observed with sublingual administration of buprenorphine and naloxone. Naltrexone, buprenorphine and naloxone are believed to compete for the same receptor sites and follow similar biotransformation pathways. The structures for naltrexone and its major metabolite, 6β-naltrexol, are very similar to naloxone. The above optimized chromatographic conditions with ammonium bicarbonate in the mobile phases were able to achieve satisfactory separation of all three analytes from the retention of naltrexone and 6β-naltrexol (up to 50 ng/ml for naltrexone and 500 ng/ml for 6β-naltrexol).

Sample preparation is also a key element in the improvement of sensitivity. A one-step protein precipitation method using methanol or acetonitrile was first investigated. Solid phase extraction was applied in many methods to clean up biological samples. Liquid-liquid extraction (LLE) can be helpful in producing a clean sample and avoiding the introduction of non-volatile materials into the column and MS system. Clean samples are essential for minimizing ion suppression and matrix effects in LC-MS/MS analysis. Different organic solvents and their mixtures in different combinations and ratios were evaluated. MTBE:hexane (2:1, v:v) was found to be optimal, because it is able to produce a clean chromatogram for a blank plasma sample and yielded the highest recovery for the analytes by a single-step liquid-liquid extraction.

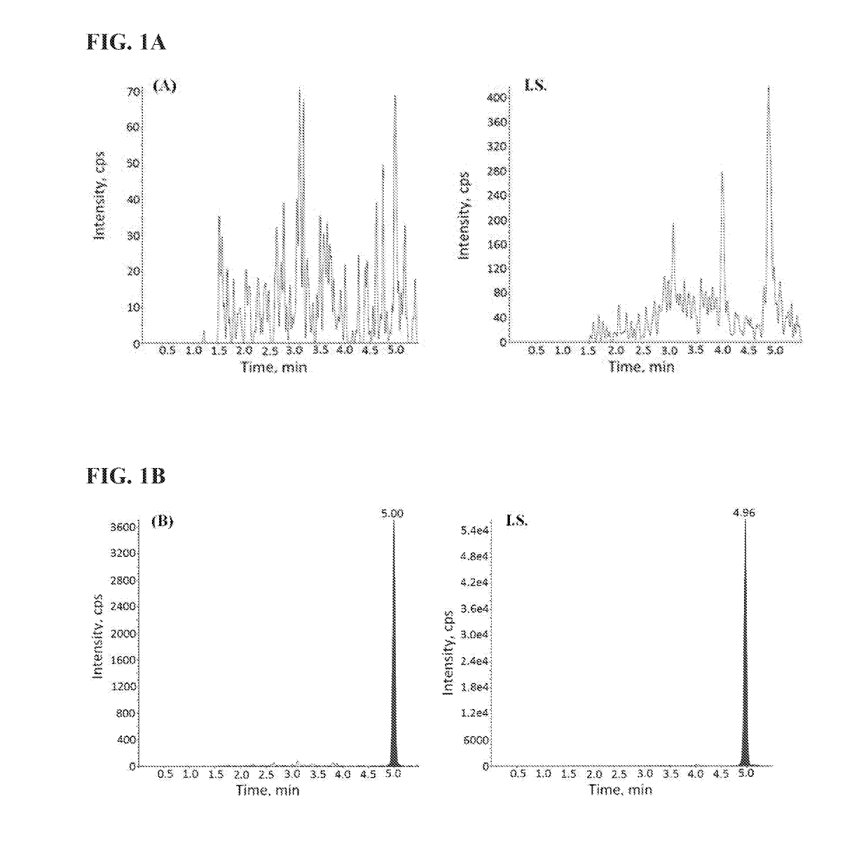

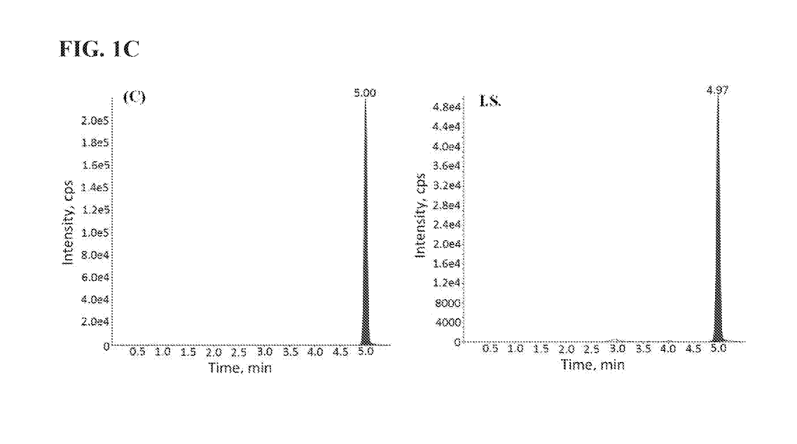

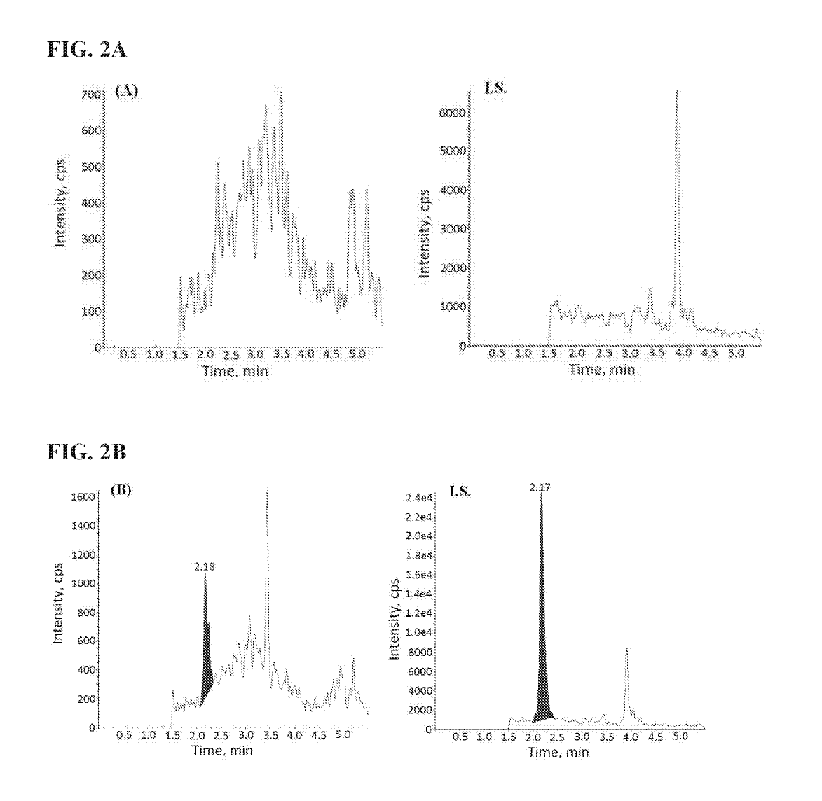

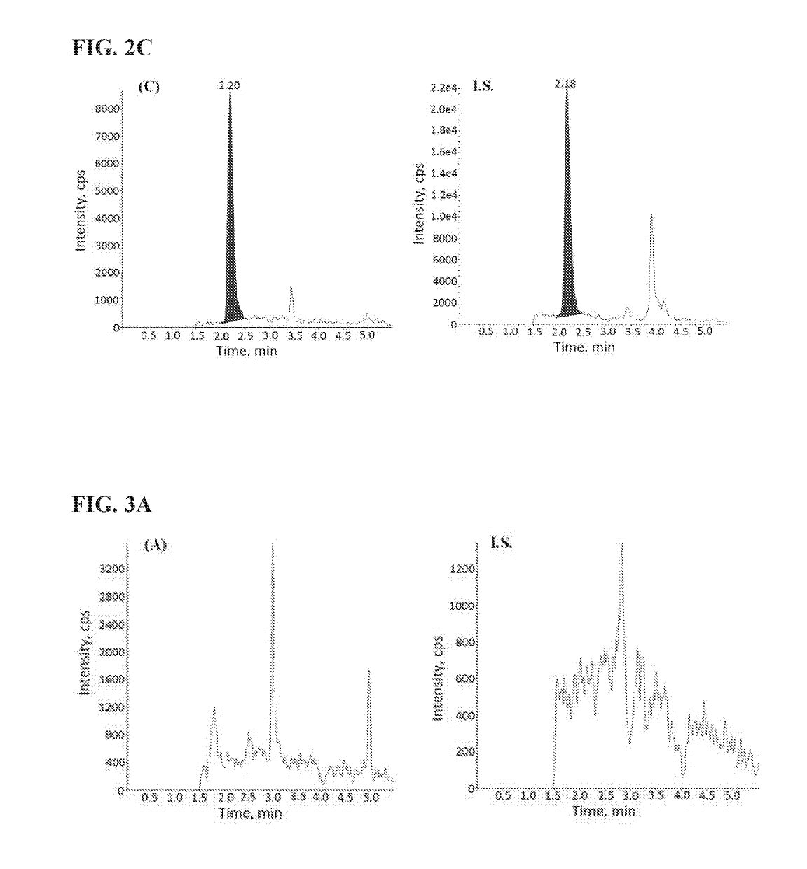

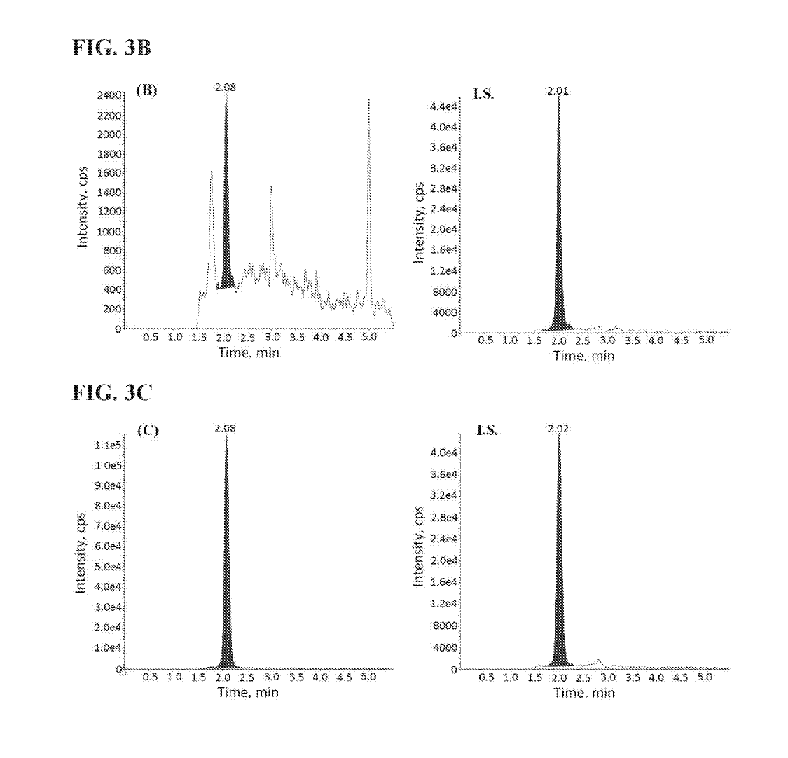

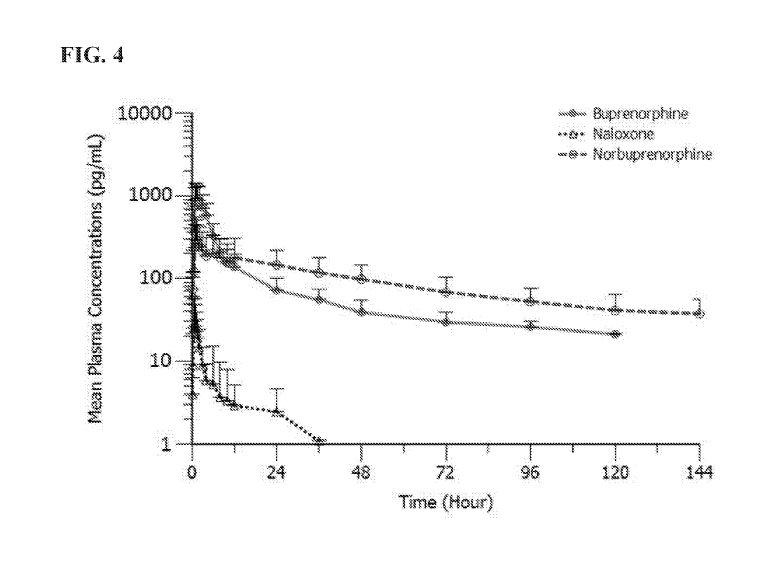

The validated methods were successfully used to quantify buprenorphine, norbuprenorphine and naloxone concentrations in healthy volunteers to evaluate the disposition of a low dose of sublingual buprenorphine (2 mg) and naloxone (0.5 mg). The representative MRM chromatograms resulting from the analysis of samples in the clinical trial are shown in FIG. 1C, FIG. 2C, and FIG. 3C. The mean (+SD) plasma concentration-time data for all three analytes are shown on a semi-log scale in FIG. 4. Buprenorphine, norbuprenorphine and naloxone were quantifiable for 120 hours, 144 hours and 36 hours, respectively. The calculated pharmacokinetic parameters for all three analytes, listed in Table 8.

TABLE 8
Summary of Geometric Mean (CV %) Plasma PK Parameter Data.

| PK Parameter | buprenorphine | norbuprenorphine | naloxone |
|---|---|---|---|
| (units) | (n =43) | (n= 8) | (n = 43) |
| Cₘₐₓ (ng/mL) | 0.953 (49.0) | 0.281 (73.5) | 0.0266 (87.5) |
| tₘₐₓ^{a} (h) | 1.50 (0.75, 6.00) | 1.00 (0.50, 6.00) | 0.75 (0.50, 6.00) |
| AUC₀₋ₗₐₛₜ (ng · h/mL) | 7.38 (37.4) | 9.71 (55.5) | 0.0681 (75.1) |
| AUC_{0-inf} (ng · h/mL) | 8.83 (31.8)^{b} | 12.4 (43.0)^{d} | 0.0772 (74.1)^{e} |
| t_{1/2} (h) | 25.9 (39.9)^{c} | 42.4 (40.3)^{c} | 2.21 (61.1)^{f} |

Abbreviations: AUC₀₋ₗₐₛₜ=area under the concentration-time curve in plasma from time zero (predose) to the time of last quantifiable concentration; AUC_{0-inf}=area under the concentration-time curve in plasma from time zero; Cₘₐₓ=maximum observed plasma concentration; PK=pharmacokinetic; tₘₐₓ=time of the maximum observed plasma concentrations; t_{1/2}=apparent terminal half-life (predose) extrapolated to infinite time. ^{a} Median (Min, Max) presented for tₘₐₓ. ^{b} n=38. ^{c} n=41. ^{d} n=27. ^{e} n=36. ^{f} n=37.

### REFERENCES

Liu et al, Journal of Pharmaceutical and Biomedical Analysis, 120:142-152 (2016) (available on-line Dec. 17, 2015); J. Chromatography, 192:222-227 (1980); J. Liquid Chromatography 4:661-680 (1981); J. Chromatography, 249:193-198 (1982): Everhar et al, Clin. Chem. 43: 2292-2302 (1997); Moody et al, J. Anal. Toxicol. 21:406-414 (1997); Vincent, et al J. Anal. Toxicol. 23:270-279 (1999); Lisi et al, J. Chromatogr. B 692:67-77 (1997); Gopal et al, Eur. J. Pharm. Biopharm. 51:147-151 (2001); Kuhlman et al, J. Anal. Toxicol. 20:229-235 (1996); Tebbett, J. Chromatogr. 347:411-413 (1985); Hackett, et al, J. Chromatogr. 374:400-404 (1986); Lagrange et al, J. Pharm. Biomed. Anal. 16:1295-1300 (1998); Ho et al, J. Chromatogr. 570:339-350 (1991); Garrett et al, J. Pharm. Sci. 74 (1985) 515-524; Kintz, J. Anal. Toxicol. 17 (1993) 443-444; Salem et al, J. Pharmacol. Toxicol. Methods 37 (1997) 75-81; Schleyer et al, J. Chromatogr. 614 (1993) 275-283; Ceccatoa et al, J. Pharm. Biomed. Anal. 32 (2003) 619-631; Regina et al, J. Chromatogr. B 939 (2013) 23-31; Wanga et al, J. Pharm. Anal. 3 (2013) 221-228; Moody et al, J. Anal. Toxicol. 21 (1997) 406-414; Polettini et al, J. Chromatogr. B 754 (2001) 447-459; Bourgogne et al, J. Pharm. Biomed. Anal. 102 (2015) 290-298; Rodriguez-Rosas et al, J. Chromatogr. B 850 (2007) 538-543; Scislowski et al, J. Anal. Toxicol. 29 (2005) 249-253; Hoja et al, J. Anal. Toxicol. 21 (1997) 160-165; Favretto et al, Rapid Commun. Mass Spectrom. 20 (2006) 1257-1265; Yuan et al, J. Chromatogr. Sep. Tech. 4 (2013), http://dx.doi.org/10.4172/2157-7064.1000174; Belse et al, J. Anal. Toxicol. 38 (2014) 438-443; Liu et al, Talanta 75 (2008)₁₉₈-204; Kronstrand et al, J. Anal. Toxicol. 27 (2003) 464-470; Tzatzarakis et al, J. Anal. Toxicol. 39 (2015) 313-320; Belivanis et al, Drug Test Anal. 6 (2014) 93-100; Kacinko et al, Anal. Chem. 80 (2008) 246-252; Liu et al, J. Anal. Toxicol. 39 (2015) 472-480; Kyle et al, Biomed. Chromatogr. (2015), http://dx.doi.org/10.1002/bmc.3460; Thomas et al, J. Sep. Sci. 33 (2010) 873-879; Grimm et al, Ther. Drug Monit. 27 (2005) 526-530; Chiang et al, Chromatographia 74 (2011) 575-583; Zhurkovich et al, Pharm. Chem. J. 48 (2015) 690-695; Moody et al, Anal. Biochem. 306 (2002) 31-39; Eckart et al, J. Chromatogr. B Analyt. Technol. Biomed. Life Sci. 1001 (2015) 1-8; Sun et al, J. Chromatogr. BAnalyt. Technol. Biomed. Life Sci. 951-952 (2014) 157-163; Martinez et al, J. Chromatogr. 528 (1990) 459-463.

What is claimed is:

1. A method for quantifying naloxone, buprenorphine, and norbuprenorphine in a human plasma sample comprising:
a. obtaining about 0.1 ml to about 1.0 ml of a plasma sample from a human;
b. performing a liquid-liquid extraction technique on the plasma sample to produce a first composition comprising naloxone, buprenorphine, and norbuprenorphine;
c. combining the first composition with a mobile phase comprising ammonium bicarbonate and methanol to produce a second composition comprising ammonium bicarbonate, methanol, naloxone, buprenorphine, and norbuprenorphine;
d. performing high pressure liquid chromatography separation on the second composition using gradient elution to produce a third composition comprising naloxone, buprenorphine, and norbuprenorphine; and
e. performing electrospray ionization mass spectrometry on the third composition to determine the concentration of the buprenorphine, naloxone, and norbuprenorphine;
thereby quantifying naloxone, buprenorphine, and norbuprenorphine in the human plasma sample.

2. A method for quantifying naloxone, buprenorphine, and norbuprenorphine in a biological sample comprising:
a. obtaining the biological sample
b. performing an extraction technique on the biological sample to produce a first composition comprising naloxone, buprenorphine, and norbuprenorphine;
c. combining the first composition with a mobile phase comprising an alcohol to produce a second composition comprising the alcohol, naloxone, buprenorphine, and norbuprenorphine;
d. performing liquid chromatography separation on the second composition using gradient elution to produce a third composition comprising naloxone, buprenorphine, and norbuprenorphine; and
e. performing electrospray ionization mass spectrometry on the third composition to determine the concentration of the buprenorphine, naloxone, and norbuprenorphine;
thereby quantifying naloxone, buprenorphine, and norbuprenorphine in the biological sample.

3. The method of claim 1 or 2, wherein the electrospray ionization mass spectrometry electrospray ionization mass spectrometry is tandem mass spectrometry.

4. The method of claim 2 or 3, wherein the liquid chromatography separation is high pressure liquid chromatography separation.

5. The method of any one of claims 1-4, wherein the biological sample is a human biological sample.

6. The method of any one of claims 1-5, wherein the biological sample is human serum, human plasma, human tissue, or human cells.

7. The method of any one of claims 1-6, wherein the biological sample is human plasma.

8. The method of any of claims 2-7, biological sample is in an amount of about 0.1 ml to about 1 ml.

9. The method of any one of claims 1-8, wherein the biological sample is in an amount of about 0.1 ml to about 0.5 ml.

10. The method of any one of claims 1-9, wherein the patient is a human.

11. The method of any one of claims 1-10, wherein the mobile phase has a varied flow rate.

12. The method of any one of claims 1-11, wherein the gradient elution has a schedule of:

| Time | Mobile Phase A | Mobile Phase B |
|---|---|---|
| (min) | (%) | (%) |
| 0.00 | 50 | 50 |
| 0.50 | 50 | 50 |
| 2.00 | 20 | 80 |
| 3.00 | 20 | 80 |
| 3.01 | 20 | 80 |
| 4.00 | 20 | 80 |
| 4.50 | 0 | 100 |
| 5.50 | 0 | 100 |
| 5.51 | 50 | 50 |
| 6.00 | 50 | 50 |
| 6.01 | 50 | 50 |

13. The method of any one of claims 1-12, wherein the gradient elution has a schedule and flow rate of:

| Time | Mobile Phase A | Mobile Phase B | Flow Rate |
|---|---|---|---|
| (min) | (%) | (%) | (mL/min) |
| 0.00 | 50 | 50 | 0.5 |
| 0.50 | 50 | 50 | 0.5 |
| 2.00 | 20 | 80 | 0.5 |
| 3.00 | 20 | 80 | 0.5 |
| 3.01 | 20 | 80 | 0.8 |
| 4.00 | 20 | 80 | 0.8 |
| 4.50 | 0 | 100 | 0.8 |
| 5.50 | 0 | 100 | 0.8 |
| 5.51 | 50 | 50 | 0.8 |
| 6.00 | 50 | 50 | 0.8 |
| 6.01 | 50 | 50 | 0.5 |

14. The method of claim 12 or 13, wherein Mobile Phase A comprises ammonium bicarbonate in water; and wherein Mobile Phase B comprises ammonium bicarbonate in methanol.

15. The method of any one of claims 12-14, wherein Mobile Phase A comprises about 0.5 mM to about 5 mM ammonium bicarbonate in water; and wherein Mobile Phase B comprises about 1 mM to about 10 mM ammonium bicarbonate in methanol.

16. The method of any one of claims 2-15, wherein the extraction technique is a solid-phase extraction technique.

17. The method of any one of claims 1-15, wherein the extraction technique is a liquid-liquid extraction technique.

18. The method of claim 1 or 17, wherein the liquid-liquid extraction comprises a C₂₋₆ linear or branched alkyl ether and a C₁₋₁₀ linear or branched alkane.

19. The method of any one of claims 1-18, wherein the mobile phase is alkali.

20. The method of any of claims 1-19, wherein the mobile phase comprises ammonium bicarbonate.

21. The method of any of claims 1-20, wherein the mobile phase comprises ammonium hydroxide.

22. The method of any of claims 1-21, wherein the mobile phase comprises ammonium bicarbonate in water.

23. The method of any of claims 1-22, wherein the mobile phase comprises ammonium bicarbonate in methanol.

24. The method of any of claims 1-23, wherein the mobile phase has a pH of about 5 to about 10.

25. The method of any of claims 1-24, wherein the mobile phase has a pH of about 6 to about 9.

26. The method of any of any one of claims 1-25, wherein the mobile phase has a pH of about 7.9.

27. The method of any one of claims 1-26, wherein quantifying naloxone, buprenorphine, and norbuprenorphine occurs simultaneously.
